(19) United States
(12) Patent Application Publication
(10) Pub. No.: US 2026/0363721 A1
(43) Pub. Date: Nov. 19, 2026

(54) SURGICAL OPTICAL FORCEPS USED FOR FOREIGN BODY ASPIRATION

(71) Applicant: American University of Beirut, Beirut (LB)

(72) Inventor: Randa Al Barazi, Beirut (LB)

(21) Appl. No.: 19/675,925

(22) Filed: May 13, 2026

Related U.S. Application Data
(60) Provisional application No. 63/804,991, filed on May 13, 2025.

(51) Int. Cl. A61B 17/29

(52) U.S. Cl. CPC A61B 17/29; A61B 2017/2939; A61B 2017/2943; A61B 2017/2947

(57) ABSTRACT

Provided herein are systems and apparatuses for Surgical Optical forceps used for foreign body aspiration.

### CROSS-REFERENCE TO RELATED APPLICATIONS

The present application claims priority to U.S. provisional application serial no. 63/804,991, filed May 13, 2025, herein incorporated by reference in its entirety.

### BACKGROUND

The invention generally relates to surgical tools, and more specifically, to optical forceps.

Rounded, flat, and smooth surface foreign bodies in the airway are particularly difficult to remove, and commercially available optical forceps are inadequate to remove them with a risk of slippage of the object further in the airway because of their acute angle while opening and closing. If the foreign body slips within the lungs, this requires highly invasive and expensive methods of removing the foreign object, including open chest surgery. Currently, basket devices used to extract these foreign bodies require at least two people, require more expertise and training to operate, are less sturdy, and are more expensive to use. As a result, there is a need for less expensive Optical forceps to increase the success rate of removing aspirated foreign bodies in a controlled manner.

In one design by the inventors in U.S. patent no. 11,103,267, the arms (designated as 180, 160, 190, and 170) were connected to the claw arms (140-150). These arms were able to rotate beyond the intended range, specifically exceeding 90 degrees. This excessive rotation caused the claws (140-150) to exhibit dual closing directions—once at an angle (less than 90 degrees) and again at 180 degrees. This uncontrolled motion led to inconsistent and unreliable performance of the claws when it is at 90 degrees.

In U.S. patent no. 11,103,267, the mechanism employed a pulling mechanism to actuate arms 220 and 210, which directly controlled the opening and closing of the claws. However, when the claws were in their fully closed position, the pulling mechanism could not achieve the necessary rotational angle to move arms 220 and 210 effectively. This resulted in insufficient force transmission, preventing smooth and complete operation.

The present invention attempts solve these problems, as well as others.

### SUMMARY OF THE INVENTION

Provided herein are systems and apparatuses for Surgical Optical forceps used for foreign body aspiration.

The methods, systems, and apparatuses are set forth in part in the description which follows, and in part will be obvious from the description, or can be learned by practice of the methods, apparatuses, and systems. The advantages of the methods, apparatuses, and systems will be realized and attained by means of the elements and combinations particularly pointed out in the appended claims. It is to be understood that both the foregoing general description and the following detailed description are exemplary and explanatory only and are not restrictive of the methods, apparatuses, and systems, as claimed.

Accordingly, it is an object of the invention not to encompass within the invention any previously known product, process of making the product, or method of using the product such that Applicants reserve the right and hereby disclose a disclaimer of any previously known product, process, or method. It is further noted that the invention does not intend to encompass within the scope of the invention any product, process, or making of the product or method of using the product, which does not meet the written description and enablement requirements of the USPTO (35 U.S.C. § 112, first paragraph) or the EPO (Article 83 of the EPC), such that Applicants reserve the right and hereby disclose a disclaimer of any previously described product, process of making the product, or method of using the product. It may be advantageous in the practice of the invention to be in compliance with Art. 53(c) EPC and Rule 28(b) and (c) EPC. All rights to explicitly disclaim any embodiments that are the subject of any granted patent(s) of applicant in the lineage of this application or in any other lineage or in any prior filed application of any third party is explicitly reserved. Nothing herein is to be construed as a promise.

### BRIEF DESCRIPTION OF THE DRAWINGS

In the accompanying figures, like elements are identified by like reference numerals among the several preferred embodiments of the present invention.

FIG. 1 is a perspective view of the surgical forceps, according to one embodiment.

FIG. 2 is an exploded view of the surgical forceps, according to one embodiment.

FIG. 3 is a top view of the surgical forceps in the open position, according to one embodiment.

FIG. 4 is a top view of the surgical forceps in the closed position, according to one embodiment.

FIG. 5 is a top view of the surgical forceps in the open position, according to one embodiment.

FIG. 6 is an exploded view of the surgical forceps, according to one embodiment.

FIG. 7 is an exploded view of the first base and second base, according to one embodiment.

### DETAILED DESCRIPTION OF THE INVENTION

The foregoing and other features and advantages of the invention are apparent from the following detailed description of exemplary embodiments, read in conjunction with the accompanying drawings. The detailed description and drawings are merely illustrative of the invention rather than limiting, the scope of the invention being defined by the appended claims and equivalents thereof.

Embodiments of the invention will now be described with reference to the Figures, wherein like numerals reflect like elements throughout. The terminology used in the description presented herein is not intended to be interpreted in any limited or restrictive way, simply because it is being utilized in conjunction with detailed description of certain specific embodiments of the invention. Furthermore, embodiments of the invention may include several novel features, no single one of which is solely responsible for its desirable attributes or which is essential to practicing the invention described herein.

The words proximal and distal are applied herein to denote specific ends of components of the instrument described herein. A proximal end refers to the end of an instrument nearer to an operator of the instrument when the instrument is being used. A distal end refers to the end of a component further from the operator and extending towards the surgical area of a patient and/or the implant.

The use of the terms “a” and “an” and “the” and similar referents in the context of describing the invention are to be construed to cover both the singular and the plural, unless otherwise indicated herein or clearly contradicted by context. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof.

Recitation of ranges of values herein are merely intended to serve as a shorthand method of referring individually to each separate value falling within the range, unless otherwise indicated herein, and each separate value is incorporated into the specification as if it were individually recited herein. The word “about,” when accompanying a numerical value, is to be construed as indicating a deviation of up to and inclusive of 10 % from the stated numerical value. The use of any and all examples, or exemplary language (“e.g.” or “such as”) provided herein, is intended merely to better illuminate the invention and does not pose a limitation on the scope of the invention unless otherwise claimed. No language in the specification should be construed as indicating any nonclaimed element as essential to the practice of the invention.

References to “one embodiment,” “an embodiment,” “example embodiment,” “various embodiments,” etc., may indicate that the embodiment(s) of the invention so described may include a particular feature, structure, or characteristic, but not every embodiment necessarily includes the particular feature, structure, or characteristic. Further, repeated use of the phrase “in one embodiment,” or “in an exemplary embodiment,” do not necessarily refer to the same embodiment, although they may.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

The words proximal and distal are applied herein to denote specific ends of components of the surgical forceps described herein. A proximal end refers to the end of the surgical forceps nearer to an operator of the instrument when the surgical forceps is being used. A distal end refers to the end of a component further from the operator and extending towards the surgical area of a patient.

### DESCRIPTION OF EMBODIMENTS

A novel optical forceps designed to extract challenging foreign bodies from the airway or lumen is disclosed herein. This surgical forceps has been designed to be compatible with commercially available bronchoscopes and scopes. In other embodiments, the surgical forceps are compatible with other scopes.

These forceps utilize a parallelogram mechanism enabling the parallel closed position of the forceps’ jaws to better secure the foreign body, particularly those rounded with smooth surfaces, during extraction. Overall, this mechanism and design allow total grasp of foreign bodies with significantly less risk of slipping complications.

The surgical forceps extract particularly challenging foreign bodies from the airway. This surgical forceps is compatible with commercially available bronchoscopes and scopes, and it requires less expertise to operate the device compared to alternate and commercially available basket devices. The surgical forceps utilize a parallelogram mechanism enabling the parallel closed position of the forceps’ jaws to better secure the foreign body without the risk of slippage. This surgical forceps provide significant cost savings by reducing the number of expensive and highly invasive procedures resulting from the slipping of foreign bodies by commercially available optical forceps. Further, the surgical forceps may be operated with a single healthcare provider, reduce the total cost of care compared to procedures that use basket devices. As a result, the surgical forceps provide an easier, cheaper, and safer retrieval solution of foreign bodies within the airway and esophagus and may be able to replace current optical forceps.

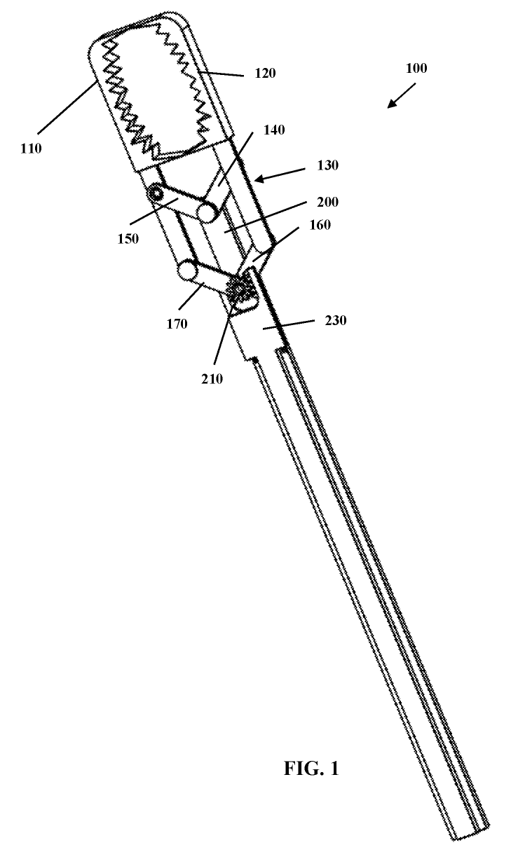

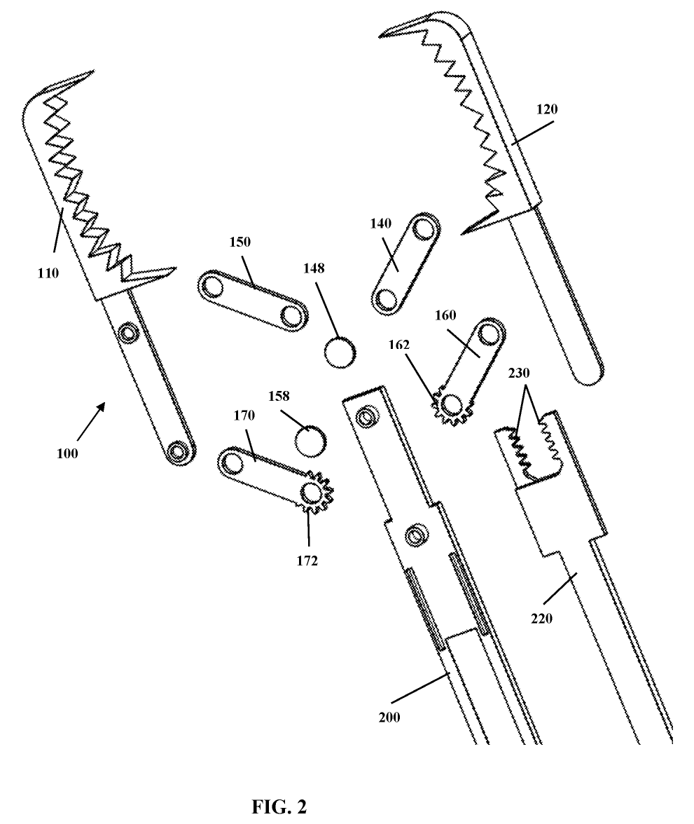

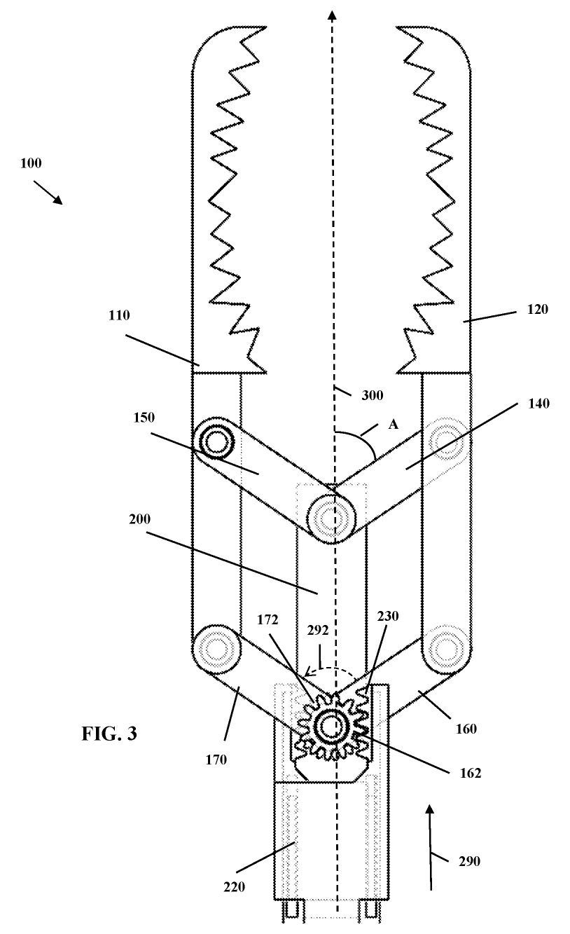

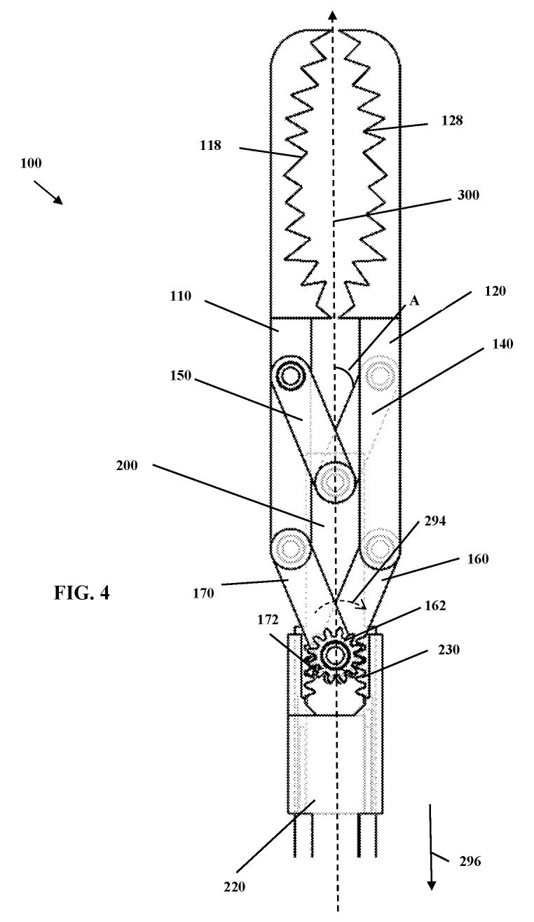

As shown in FIGS. 1 and 2, the surgical forceps 100 comprise a first clamp 110 and a second clamp 120 operably coupled to a parallelogram mechanism 130 to move the first clamp 110 and the second clamp 120 from a nearly parallel open position to a nearly parallel closed position, as shown in FIGS. 3 and 4. The parallelogram mechanism 130 comprises a first link 140 operably coupled to a first base 200 and a second link 150 operably coupled to the first base 200. A third link 160 is operably coupled to a gear 210 and a fourth link 170 is operably coupled to the gear 210, whereby rotation of the gear 210 translates the first clamp 110 and the second clamp 120 to the open and closed position. Rotation of the gear 210 is accomplished by the distal movement of the second base 220. The third link 160 includes a third gear 162 on the distal end and the fourth link 170 includes a third gear 172 on the distal end, wherein the third gear 162 and the fourth gear 172 rotate with the gear 210 upon distal movement of the second base 220. The second base 220 includes a rack spur 230 that mates with the third gear 162 and the fourth gear 172. A plurality of tabs 148, 158 are used to secure the first link 140, second link 150, third link 160, and fourth link 170 the first clamp 110 and the second clamp 120. In one embodiment, the gear 210 is fixedly coupled to the fourth link 170 and the third link 160. In another embodiment, the gear 210 is two separate gears, wherein the fourth link 170 includes a gear and the third link 160 includes a gear, as further detailed below.

As shown in FIG. 3, the surgical forceps 100 are in the open position. The second base 220 is distally translated in the direction of 290, which distally moves the rack spur 230 causing the third gear 162 and the fourth gear 172 to rotate in the direction of 292. The third link 160 is rotatably coupled with the second clamp 120. The fourth link 170 is rotatably coupled with the first clamp 110. The rotation of the third gear 162 and the fourth gear 172 cause the first clamp 110 and the second clamp 120 to move to the closed position in parallel, as shown in FIG. 4. The first clamp 110 includes a jaw portion 118 and the second clamp 120 includes a jaw portion 128, wherein the jaw portions 118, 128 secure foreign matter or material. Since the fourth link 170 is parallel to the second link 150 and third link 160 is parallel to the first link 140, they assist in the movement of the first clamp 110 and second clamp 120 to the closed position in parallel.

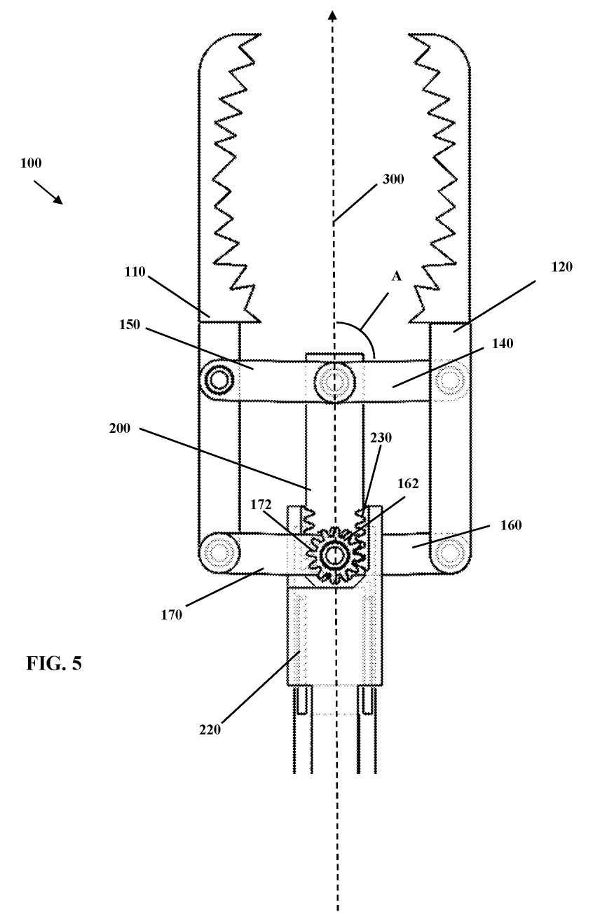

The lengths and geometry of the first link, second link, third link, and fourth link have been modified to restrict their rotational movement within a predefined range. Specifically, the first link, second link, third link, and fourth link operate within a minimum angle A of 0 degrees and a maximum angle of 90 degrees with respect to the longitudinal axis 300 of the surgical forceps, as shown in FIG. 5. This constraint ensures precise and consistent motion of the first clamp and the second clamp, eliminating the dual closing directions and providing a more reliable mechanism. To move from the closed position back to the open position, the second base 220 is proximally translated in the direction of 296 to proximally move the rack spur 230 causing the third gear 162 and the fourth gear 172 to rotate in the direction of 294, as shown in FIG. 4. The rotation of the third gear 162 and the fourth gear 172 cause the first clamp 110 and the second clamp 120 to move to the open position in parallel.

The parallelogram mechanism 130 operates as a rack and pinion mechanism to enhance control and efficiency. Each clamp is now independently operated by a dedicated rack and pinion system. However, the clamps are mechanically synchronized, ensuring simultaneous and coordinated movement of both pinions. This design allows for smoother operation, greater force transmission, and precise control of the opening and closing motions of the claws. These improvements result in a mechanism that is more reliable, precise, and efficient in operation, addressing the limitations of the previous design while enhancing overall performance.

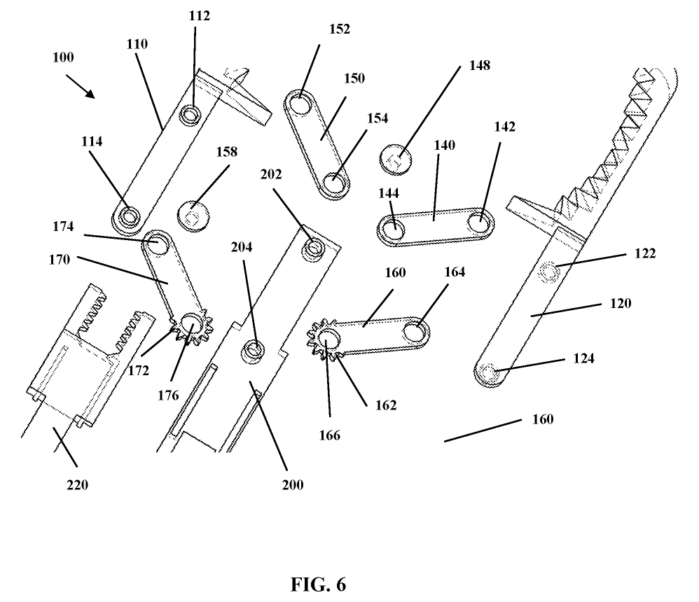

As shown in FIG. 6, the first clamp 110 includes a first coupling 112 and a second coupling 114 and the second link 150 includes a first opening 152 and a second opening 154, wherein the first opening 152 rotatably couples with the first coupling 112. The first base 200 include a first coupling 202 and a second coupling 204, wherein the second opening 154 rotatably couples with the first coupling 202. The first link 140 includes a first opening 142 and second opening 144, wherein the second opening 144 rotatably couples with the first coupling 202 of the first base 200. The second clamp 120 includes a first coupling 122 and a second coupling 124, wherein the first opening 142 of the first link 140 rotatably couples with the first coupling 122 of the second clamp 120. The tab 148 may secure the first link 140 and the second link 150 to the first base 200. The third link 160 includes a first opening 164 and a second opening 166. The first opening 164 rotatably couples with the second coupling 124 of the second clamp 120. The second opening 166 rotatably couples with the second coupling 204 of the first base 200. The fourth link 170 includes a first opening 174 and second opening 176, wherein the first opening 174 rotatably couples with the second coupling 114 of the first clamp 110. The tab 158 may secure the fourth link 170 to the first clamp 110. The second opening 176 of the fourth link 170 rotatably couples with the second coupling 204 of the first base 200.

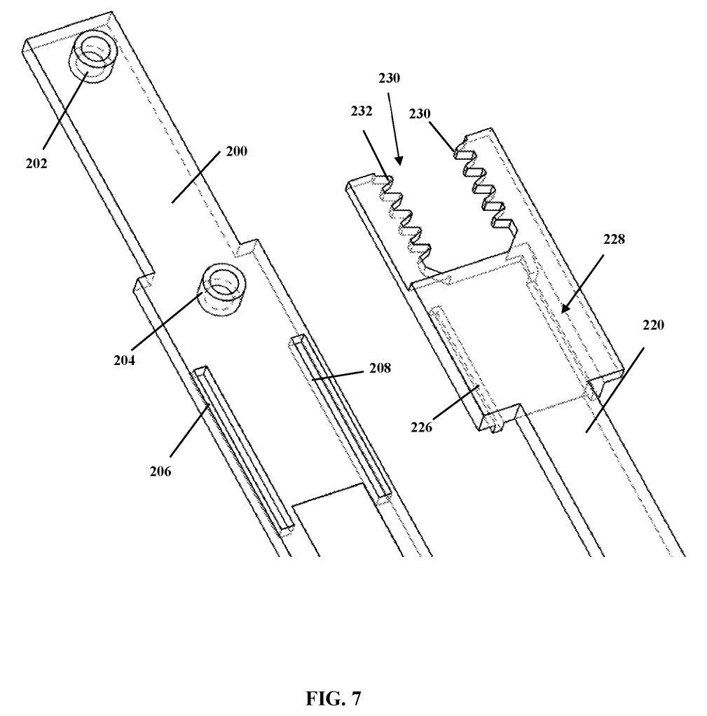

As shown in FIG. 7, the first base 200 includes a longitudinal slide portion including top slide 208 and a bottom slide 206. The second base 220 includes a first inner slide 228 and a second inner slide 226, wherein the first inner slide 228 longitudinally fits within the top slide 208 and the second inner slide 226 longitudinally fits within the bottom slide 206. The longitudinal slide portion allows the second base 220 to longitudinally move along the first base 200 from the open position to the closed position and from the closed position to the open position. As such, the top slide 208 and the bottom slide 206 include stop portions to prevent additional longitudinal movement of the second base 220. And the rack spur 230 includes a top spur 232 and a bottom spur 234, wherein the top spur 232 rotatably engages with the gear 172 on the fourth link 170 and the bottom spur 234 rotatably engages with the gear 162 on the third link 160.

As can be understood by one skilled in the art, the surgical forceps 100 and/or any of its components may have any size, shape, length, thickness, height, weight, or any other parameters. Such parameters may be selected by the surgeon (or other qualified professional) for performance of specific procedures. Further, the surgical forceps and/or any of its components may be manufactured from metal, plastic, synthetic material, or other suitable materials, or any combination thereof. In one embodiment, the surgical forceps 100 is composed of a metal alloy, titanium, nitinol, or stainless steel, or alternatively, any medical grade composite or ceramic.

In some embodiments, various lengths and configurations may also include various features to accommodate different applications for the surgical forceps. The surgical forceps can be constructed of various materials to aid in radio translucency, strength, flexibility, and integration with anatomy, etc.

### Examples

The following examples are put forth so as to provide those of ordinary skill in the art with a complete disclosure and description of how the compounds, compositions, articles, devices and/or methods claimed herein are made and evaluated and are intended to be purely exemplary of the invention and are not intended to limit the scope of what the inventors regard as their invention. However, those of skill in the art should, in light of the present disclosure, appreciate that many changes can be made in the specific embodiments which are disclosed and still obtain a like or similar result without departing from the spirit and scope of the invention.

Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example: Improvement of surgical forceps over the art

All publications and patent applications mentioned in this specification are herein incorporated by reference to the same extent as if each individual publication or patent application was specifically and individually indicated to be incorporated by reference.

While the invention has been described in connection with various embodiments, it will be understood that the invention is capable of further modifications. This application is intended to cover any variations, uses or adaptations of the invention following, in general, the principles of the invention, and including such departures from the present disclosure as, within the known and customary practice within the art to which the invention pertains.

What is claimed is:

1. Surgical forceps comprising: a first clamp and a second clamp operably coupled to a parallelogram mechanism to move the first clamp and the second clamp from a nearly parallel open position to a nearly parallel closed position; the parallelogram mechanism comprises a first link operably coupled to a first base and a second link operably coupled to the first base; a third link is operably coupled to a gear and a fourth link is operably coupled to the gear, whereby rotation of the gear translates the first clamp and the second clamp to the open and closed position; rotation of the gear is translated by the distal movement of the second base; the third link includes a third gear on the distal end and the fourth link includes a third gear on the distal end, wherein the third gear and the fourth gear rotate with the gear upon distal movement of the second base; the second base includes a rack spur that mates with the third gear and the fourth gear.

2. The surgical forceps of claim 1, further comprising a plurality of tabs to secure the first link, the second link, the third link, and the fourth link the first clamp and the second clamp.

3. The surgical forceps of claim 2, wherein the gear is fixedly coupled to the fourth link and the third link.

4. The surgical forceps of claim 3, wherein the gear is two separate gears, wherein the fourth link includes a gear and the third link includes a gear.

5. The surgical forceps of claim 4, wherein the second base is distally translated in the direction of, which distally moves the rack spur causing the third gear and the fourth gear to rotate in the counter clockwise direction; the third link is rotatably coupled with the second clamp; the fourth link is rotatably coupled with the first clamp.

6. The surgical forceps of claim 5, wherein the rotation of the third gear and the fourth gear cause the first clamp and the second clamp to move to the closed position in parallel.

7. The surgical forceps of claim 6, wherein the first clamp includes a jaw portion and the second clamp includes a jaw portion, wherein the jaw portions secure foreign matter or material.

8. The surgical forceps of claim 7, wherein the fourth link is parallel to the second link and third link is parallel to the first link to assist in the movement of the first clamp and second clamp to the closed position in parallel.

9. The surgical forceps of claim 8, wherein the first link, the second link, the third link, and the fourth link operate within a minimum angle A of 0 degrees and a maximum angle of 90 degrees with respect to the longitudinal axis of the surgical forceps.

10. The surgical forceps of claim 9, wherein the second base is proximally translated to proximally move the rack spur causing the third gear and the fourth gear to rotate in the direction of, in order to move from the closed position to the open position; and the rotation of the third gear and the fourth gear cause the first clamp and the second clamp to move to the open position in parallel.

11. The surgical forceps of claim 10, wherein the parallelogram mechanism operates as a rack and pinion mechanism to mechanically synchronize the first clamp and the second clamp.

12. The surgical forceps of claim 11, wherein the first clamp includes a first coupling and a second coupling and the second link includes a first opening and a second opening, wherein the first opening rotatably couples with the first coupling; the first base include a first coupling and a second coupling, wherein the second opening rotatably couples with the first coupling; the first link includes a first opening and second opening, wherein the second opening rotatably couples with the first coupling of the first base; the second clamp includes a first coupling and a second coupling, wherein the first opening of the first link rotatably couples with the first coupling of the second clamp; the tab secures the first link and the second link to the first base; the third link includes a first opening and a second opening; the first opening rotatably couples with the second coupling of the second clamp; the second opening rotatably couples with the second coupling of the first base; the fourth link includes a first opening and second opening, wherein the first opening rotatably couples with the second coupling of the first clamp; the tab secures the fourth link to the first clamp; the second opening of the fourth link rotatably couples with the second coupling of the first base.

13. The surgical forceps of claim 12, wherein the first base includes a longitudinal slide portion including top slide and a bottom slide; the second base includes a first inner slide and a second inner slide, wherein the first inner slide longitudinally fits within the top slide and the second inner slide longitudinally fits within the bottom slide; the longitudinal slide portion allows the second base to longitudinally move along the first base from the open position to the closed position and from the closed position to the open position.

14. The surgical forceps of claim 13, wherein the top slide and the bottom slide include stop portions to prevent additional longitudinal movement of the second base.

15. The surgical forceps of claim 14, wherein the rack spur includes a top spur and a bottom spur, wherein the top spur rotatably engages with the gear on the fourth link and the bottom spur rotatably engages with the gear on the third link.
